(19) United States
(12) Patent Application Publication
(10) Pub. No.: US 2026/0056174 A1
(43) Pub. Date: Feb. 26, 2026

(54) CALIBRATION SYSTEM AND CALIBRATION METHOD FOR NDIR GAS SENSORS

(71) Applicants: SHENZHEN UNITENSE INNOVATION ELECTRONICS COMPANY LIMITED, SHENZHEN (CN); THE HONG KONG POLYTECHNIC UNIVERSITY, HONG KONG (CN)

(72) Inventors: SHIMEI CHEN, SHENZHEN (CN); SIU WING OR, HONG KONG (CN); QUANYUAN CHEN, SHENZHEN (CN); YAOGUANG LI, SHENZHEN (CN)

(21) Appl. No.: 18/812,089

(22) Filed: Aug. 22, 2024

(51) Int. Cl. G01N 33/00; G01N 21/01; G01N 21/3504

(52) U.S. Cl. CPC G01N 33/0008; G01N 21/3504; G01N 2021/0131; G01N 2201/1211; G01N 2201/12784

(57) ABSTRACT

The present invention relates to the technical field of sensor calibration, particularly a calibration system and a calibration method for NDIR gas sensors. The calibration system comprises: a client-server computer network with calibration software, a relay module, a gas source group, a mass flow controller, a high and low temperature chamber, a gas analyzer, and a calibration tooling rack group for implementing broadcasting-style calibration. The calibration method consists of: a Lambert-Beer weighted concentration calculation mode combined with an adaptive piecewise linear temperature compensation mode. Integrating the calibration system and the calibration method can effectively streamline the calibration process, improve the calibration accuracy, and reduce the calibration time and cost of the NDIR gas sensors. It can, therefore, conveniently realize a simultaneous calibration of many NDIR gas sensors for mass production and enable a greener production environment with a higher degree of carbon neutrality.

### TECHNICAL FIELD

The present invention relates to the technical field of sensor calibration, particularly a calibration system and a calibration method for NDIR (Non-Dispersive InfraRed) gas sensors.

### BACKGROUND

The NDIR gas sensors are widely used in the fields of indoor and outdoor air quality monitoring, methane gas leakage monitoring, DCV (demand control ventilation) process and HVAC (heating, ventilation, and air conditioning) system monitoring, smart grid power system condition monitoring, medical equipment and surgical operations monitoring, agricultural greenhouse monitoring, transportation cabin and cargo monitoring, and the like because of their advantages of insusceptibility to harmful gas and poisoning, fast response and recovery times, high accuracy, good stability, and long lifespan.

However, the calibration of the NDIR gas sensors, which has been disclosed till now, still has disadvantages in two aspects: calibration system and calibration method.

The traditional calibration system is heavy in calculation load, time-consuming in operation, and high consumption in energy and cost. The frequent data acquisition and massive data processing tasks by a computer are the fundamental reasons for heavy calculation load, prolonged operation time, and high energy and cost consumption. The computer first needs to read a large number of calibration data, including calibration concentration level, calibration temperature level, and the corresponding voltage signals (referred to as concentration voltage and temperature voltage henceforth) from each NDIR gas sensor, then calculate the calibration concentration coefficient and the calibration temperature compensation coefficient, next send the calculated calibration coefficients to each NDIR gas sensor, and last wait for each of the responses. The calibration system needs to take a long time to operate, imposing significant payloads on mass production.

The traditional calibration method has deficiencies in its concentration calculation mode and temperature compensation mode, elaborated as follows:

The existing concentration calculation mode uses a conventional fitting algorithm (such as polynomial curve fitting with one variable, surface fitting, and piecewise linear fitting) with many calibration data, making it challenging to adjust particular calibration points (usually formed by calibration concentration level and calibration temperature level), inducing significant calculation errors and, hence, calibration errors within low concentration ranges. The calculation/calibration accuracy cannot be guaranteed if the number of calibration data/points is reduced. For example, the polynomial curve fitting with one variable algorithm used in the patent literature with the publication number CN110411970A necessitates collecting substantial calibration data within the measurement range to achieve the desired calibration accuracy. For example, the surface fitting algorithm employed in the patent literature with the publication number CN112903617A necessitates 60 calibration points, consisting of 12 calibration concentration levels for each of the 5 calibration temperature levels, during calibration. For example, the piecewise linear fitting algorithm used in the patent literature with the publication number CN115468925A requires 24 calibration points, comprising 6 calibration concentration levels for each of the 4 calibration temperature levels, during calibration. Still, these conventional fitting algorithms cannot satisfy the increasing challenge of removing errors within low concentration ranges. For the same 50 ppm fitting error, when the reading is 500 ppm, the resulting relative error of reading is 10%, and when the reading is 5,000 ppm, the resulting relative error of reading is 1%. Alternatively, a dedicated fitting algorithm based on the Lambert-Beer curve fitting, as presented in Alphasense's application note AAN 204-02, can be employed for the concentration calculation mode of the NDIR sensor calibration. However, as the space is usually ample in a realistic batch calibration environment (for example, 1 to 10 m³), the requirement of acquiring the initial signal intensity at zero level of gas concentration adds an extra 3 to 10 hours in time for reducing the gas concentration to be measured to zero by consuming additional energy and pure nitrogen. Although a high-dimensional concentration calculation mode based on a genetic or neural network algorithm (such as patent publication number CN110057773A) shows better accuracy, its super-high computing power requirements limit popularization and industrial use.

The existing temperature compensation mode is incapable of describing the complicated, non-linear, and non-monotonous temperature characteristics of the NDIR gas sensors (as shown in FIG. 1). The reasons that the temperature characteristics of the NDIR gas sensors become complicated, non-linear, and non-monotonous can be attributed to four aspects: First, the temperature drift of different electronic components is non-linear and inconsistent. Second, individual differences exist in the thermal deformation of the same kind of optical devices, and the thermal deformation effect on the signal strength is non-linear. Third, individual differences exist in the temperature drift of the same electronic components (such as operational amplifiers). Fourth, the absorption coefficient of the gas and that of the filter change with the temperature at non-linear change rates. The conventional hardware-based linear temperature compensation mode (patent publication number CN107192685A) cannot be fully applied to the complex, non-linear, and non-monotonous temperature characteristics of the NDIR gas sensors. The existing temperature compensation mode using a linear fitting algorithm (patent publication number CN113252597) cannot be fully applied to the non-linear temperature characteristics of the NDIR gas sensors. The existing temperature characteristic curve fitting algorithm (patent publication number CN115468925A) is not always applicable to the non-monotonous temperature characteristics of the NDIR gas sensors. The existing temperature piecewise linear fitting algorithm (patent publication number CN114993474A) has such a disadvantage that the temperature compensation coefficient is fixed and unchangeable with the concentration.

Therefore, developing a more suitable calibration system and a more suitable calibration method is an effective way to streamline the calibration process, improve the calibration accuracy, and reduce the calibration time and cost of the NDIR gas sensors, thereby realizing a simultaneous calibration of many NDIR gas sensors for mass production and enabling a greener production environment with a higher degree of carbon neutrality.

### SUMMARY

In view of this, the present invention provides a calibration system and a calibration method for the NDIR gas sensors. The calibration system includes a client-server computer network with calibration software and other functional devices for implementing broadcasting-style calibration. The calibration method comprises a Lambert-Beer weighted concentration calculation mode and an adaptive piecewise linear temperature compensation mode. Integrating the calibration system and the calibration method can effectively streamline the calibration process, improve the calibration accuracy, and reduce the calibration time and cost of the NDIR gas sensors. It can, therefore, conveniently realize a simultaneous calibration of many NDIR gas sensors for mass production and enable a greener production environment with a higher degree of carbon neutrality.

In order to achieve the above purpose, the present invention adopts the following technical solution:
In the first aspect, the present invention provides a calibration system for the NDIR gas sensors, which comprises: a client-server computer network with calibration software, a relay module, a gas source group, a mass flow controller, a high and low temperature chamber, a gas analyzer, and a calibration tooling rack group; the client-server computer network is installed with calibration software (developed according to the calibration method, further elaborated upon later) to control and execute broadcasting-style calibration; the calibration tooling rack group is installed in the high and low temperature chamber, and a plurality of calibration tooling plates are installed on the calibration tooling rack group; the NDIR gas sensors to be calibrated are detachably installed on the corresponding calibration tooling plates and connected with the corresponding calibration tooling plates; and a storage medium is arranged inside each NDIR gas sensor and used for storing calibration data;
Each gas source in the gas source group is connected with the high and low temperature chamber, respectively, through a gas path, and each gas path is provided with a pressure sensor and a solenoid valve; the relay module is connected with the pressure sensor and the solenoid valve, respectively; and the mass flow controller is connected with the solenoid valve through the gas path for adjusting the size of the gas flow;
The gas analyzer is connected with the high and low temperature chamber for collecting the actual gas concentration to be measured in the high and low temperature chamber;

The client-server computer network with calibration software is connected with the relay module, the mass flow controller, the high and low temperature chamber, the gas analyzer, and each of the calibration tooling plates to realize control or data exchange.

Further, a calibration tooling rack group having at least two calibration tooling racks is accommodated in the high and low temperature chamber; each of the calibration tooling racks has multiple layers; each layer is provided with at least two connector plates; each connector plate is connected through a golden finger and loaded with a plurality of calibration tooling plates; each calibration tooling plate has a plurality of installation numbers; and each of the NDIR gas sensors is installed on each of the installation numbers one by one.

Further, a plurality of tooling power supplies are also installed on the calibration tooling racks, and each of the tooling power supplies powers the plurality of NDIR gas sensors.

Further, the calibration system further comprises a 24 V switch power supply, and the 24 V switch power supply powers the pressure sensor, the solenoid valve, the relay module, and the mass flow controller.

Further, the gas source group comprises a cylinder of the gas to be measured, a compressed air source, and a nitrogen cylinder.

Further, each NDIR gas sensor comprises an optical sensing cell, an infrared light source, a detector, a bandpass amplifier, a follower, an ADC (Analog-to-Digital Converter) module, an MCU (MicroController Unit), and a driving unit;

The optical sensing cell is provided with an air inlet and an air outlet; the infrared light source and the detector are relatively arranged in the optical sensing cell; the driving unit drives the infrared light source to emit infrared light under the control of the MCU; and the optical sensing cell reflects the infrared light emitted by the infrared light source for entering the detector;

A thermopile device and an NTC (Negative Temperature Coefficient) thermistor are arranged inside the detector, and two pins are led out, i.e., a thermopile pin PIN1 and an NTC thermistor pin PIN2; the thermopile PIN1 is connected with the ADC module through the bandpass amplifier, and the NTC thermistor pin PIN2 is connected with the ADC module through the follower; the MCU is connected with the ADC module for controlling the sampling of the ADC module; and the ADC module measures the peak-to-peak value of a voltage signal outputted by the bandpass amplifier, which is referred to as the concentration voltage Vₚₚ; the variation in V_{PP} indicates changes in gas concentration; additionally, the ADC module captures the voltage signal outputted by the follower as the temperature voltage V_{NTC}, where fluctuations in V_{NTC} correspond to variations in temperature;

The MCU is connected with the calibration tooling plates and the client-server computer network with calibration software successively through a digital communication interface, and receives data capturing instructions, data saving instructions, and the actual gas concentration to be measured in the high and low temperature chamber, broadcasted by the client-server computer network with calibration software;

The storage media are located inside the MCU, and store the concentration voltage V_{PP}, the temperature voltage V_{NTC}, and the actual gas concentration to be measured under the control of the MCU.

In the second aspect, the present invention provides a calibration method for the NDIR gas sensors, which is applicable to the calibration system of the NDIR gas sensors, comprising the following steps:
determining m calibration temperature levels and n calibration concentration levels according to the working temperature and the measurement range of the NDIR gas sensors;
capturing, by the NDIR gas sensors, calibration data (xᵢⱼ, yᵢⱼ, zᵢⱼ) at each calibration concentration level at any calibration temperature level, wherein i=1, 2, 3, . . . , m; j=1, 2, 3, 4, . . . , n; xᵢⱼ is the concentration voltage V_{PP} currently outputted by the NDIR gas sensors; zᵢⱼ is the temperature voltage V_{NTC} currently outputted by the NDIR gas sensors; and yᵢⱼ is the actual gas concentration to be measured in the high and low temperature chamber collected by the gas analyzer;
broadcasting and transmitting, by the client-server computer network with calibration software, the data saving instructions to each NDIR gas sensor, and saving, by the NDIR gas sensors, the calibration data to the internal storage media;
extracting, by the NDIR gas sensors, (xᵢⱼ, yᵢⱼ) from the calibration data with the calibration temperature level of T₁, wherein i=1; and j=1, 2, 3, 4, . . . , n;
fitting (xᵢⱼ, yᵢⱼ) as a curve, denoted as y₁(x), calculating the residual sum E of squares of (xᵢⱼ, yᵢⱼ) according to the principle of a weighted least square curve fitting and setting a weight wᵢⱼ, wherein i=1; and j=1, 2, 3, 4, . . . , n;
solving fitting coefficients α, β, and x₀ corresponding to the fitting curve y₁(x) and corresponding temperature t₁;
solving, by the NDIR gas sensors, fitting curves y₂(x), y₃(x), . . . , yₘ(x) and corresponding temperatures t₂, t₃, . . . , tₘ successively at the calibration temperature levels of T₂, T₃, . . . , Tₘ according to the same process;
dividing the temperatures t₁, t₂, t₃, . . . , to into m−1 temperature intervals, which are: [tₘ, tₘ₋₁], . . . , [t₃, t₂], [t₂, t₁];
acquiring current data Vₚₚ=xᵣ and V_{NTC}=Zᵣ by the NDIR gas sensors, and converting zᵣ into temperature, denoted as tᵣ, tᵣ∈[tₘ, t₁];
confirming the temperature interval where tᵣ is located, and selecting adjacent fitting curves to calculate the gas concentration to be measured yᵣ and the temperature compensation coefficient K.

Further, the solving process of the fitting coefficients α, β, and x₀ corresponding to the fitting curve y₁(x) is as follows:
calculating the residual sum E of squares of (xᵢⱼ, yᵢⱼ) according to the following formula:

$$E = {\sum }_{j = 1}^{n} {{w}_{ij} [ y ( {x}_{ij} ) - {y}_{ij} ]}^{2} = {\sum }_{j = 1}^{n} {{w}_{ij} [ {( \frac{- ln ( {x}_{ij} / {x}_{0} )}{α} )}^{\frac{1}{β}} - {y}_{ij} ]}^{2}$$

according to an extremum principle, the first-order partial derivative of α, β, and x₀ in the above formula is 0, so that the residual sum E of squares is minimum, wherein i=1; and j=1, 2, 3, 4, . . . , n;

solving the fitting coefficients α, β, and x₀ of the fitting curve y₁(x) for implementing the Lambert-Beer weighted concentration calculation mode.

Further, the calculation of temperature t₁ is as follows:
at the calibration temperature level T₁, calculating the average value of the temperature voltage zᵢⱼ outputted by the NDIR gas sensors at each calibration concentration level, denoted as

$${\overline{z}}_{1} , \overline{{z}_{1}} = \frac{1}{n} {\sum }_{j = 1}^{n} {z}_{ij} ,$$

wherein i=1; and j=1, 2, 3, 4, . . . n;
converting z̅₁ into temperature, denoted as t₁, in ° C.

Further, the Lambert-Beer weighted concentration calculation mode combined with the adaptive piecewise linear temperature compensation mode is implemented as follows:
when the temperature tᵣ∈[tₘ, tₘ₋₁], the fitting curves yₘ₋₁(x) and yₘ(x) are selected, xᵣ is substituted into the curves to calculate yₘ₋₁(xᵣ) and yₘ(xᵣ), respectively, and the temperature compensation coefficient K and the gas concentration to be measured yᵣ are calculated according to the following formulas:

$$K = \frac{[ {y}_{m - 1} ( {x}_{r} ) - {y}_{m} ( {x}_{r} ) ]}{{t}_{m - 1} - {t}_{m}} \\ {y}_{r} = K ( {t}_{r} - {t}_{m} ) + {y}_{m} ( {x}_{r} ) .$$

According to the above technical solution, compared with the prior art, the present invention has the following beneficial effects:

1. The calibration tooling rack group of the present invention can realize simultaneous calibration of a large number of NDIR gas sensors, and the client-server computer network with calibration software broadcasts the instruction data to each NDIR gas sensor, which can reduce the number of data interactions and data processing tasks of the client-server computer network with calibration software.

The high and low temperature chamber can provide stable calibration temperature levels in the calibration process of the NDIR gas sensors. The gas analyzer can detect the actual gas concentration to be measured in the high and low temperature chamber to provide a reference basis for the concentration calibration of the NDIR gas sensors. The pressure sensor is installed on each gas source pipeline in the gas source group to monitor whether the pressure of the gas source is satisfactory in real-time to ensure that the corresponding gas source is smoothly transmitted to the high and low temperature chamber. At the same time, the compressed air source and the nitrogen cylinder are arranged so that nitrogen or air can be injected into the high and low temperature chamber, so as to adjust the gas concentration to be measured in the high and low temperature chamber to facilitate the calibration accuracy of the NDIR gas sensors.

2. The client-server computer network with calibration software transmits the data-capturing instructions and the data-saving instructions in sequence by way of broadcasting, which takes a short time and has a low calculation load. After receiving the instructions, the NDIR gas sensors capture the calibration data and save the data in the internal storage media, thereby reducing the number of data interactions for the client-server computer network with calibration software. The NDIR gas sensors independently calculate the fitting coefficients and perform concentration calculation and temperature compensation, thereby reducing the data processing tasks of the client-server computer network with calibration software.

3. Through the calibration method of the present invention, when the calibration points are reduced appropriately, the calibration accuracy of the NDIR gas sensors is not significantly reduced, so the calibration efficiency is significantly improved.

4. The present invention adopts the Lambert-Beer weighted calibration mode, and reduces the number of calibration points under the premise of satisfying the calibration accuracy compared with the conventional Lambert-Beer fitting. At the same time, when the NDIR gas sensors are calibrated, I₀ in the conventional Lambert-Beer fitting I=I₀·e^{(-KLC)} needs to be sampled when the ambient gas concentration to be measured is 0. In the realistic batch calibration environment, the space is large (e.g., 1 to 10 m³), it takes an additional 3 to 10 hours to reduce the gas concentration to be measured to 0, and it uses a lot of energy and pure nitrogen. The present invention adopts the Lambert-Beer weighted concentration calculation mode with adaptive piecewise linear temperature compensation, and I₀ is a derived value of an initial signal intensity obtained by fitting calculation, which reduces the calibration time. Compared with the conventional Lambert-Beer fitting, the present invention does not need to reduce the gas concentration to be measured to zero, thereby reducing the calibration cost and improving the production efficiency.

5. Because the interval between adjacent fitting curves is unequal and the temperature compensation coefficient K is related to the concentration and temperature intervals, in the calibration method of the present invention, the temperature compensation coefficient is adaptively adjusted with the change of the concentration and temperature intervals in view of the temperature characteristics of complexity, non-linearity, and non-monotonicity of the NDIR gas sensors, to reduce the influence of the temperature on the calibration accuracy of the NDIR gas sensors.

### DESCRIPTION OF DRAWINGS

To more clearly describe the technical solutions in the embodiments of the present invention or in the prior art, the drawings required to be used in the description of the embodiments or the prior art will be simply presented below. Apparently, the drawings in the following description are merely the embodiments of the present invention, and for those ordinary skilled in the art, other drawings can also be obtained according to the provided drawings without contributing creative labor.

FIG. 1 is a temperature characteristic curve of NDIR gas sensors.

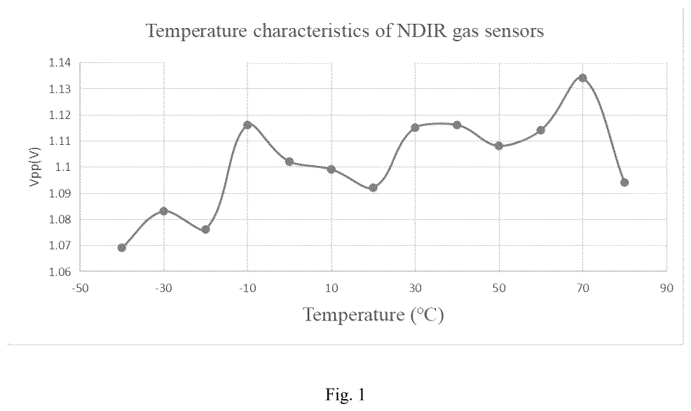

FIG. 2 is a structural schematic diagram of a calibration system of NDIR gas sensors provided by the present invention.

FIG. 3 is a structural schematic diagram of an NDIR gas sensor provided by the present invention.

FIG. 4 is a main flow chart of the calibration method of NDIR gas sensors provided by the present invention.

FIG. 5 is a detailed flow chart for capturing calibration data provided by the present invention.

FIG. 6 is a detailed flow chart for solving the Lambert-Beer curve fitting coefficients with the weighted least square curve fitting algorithm.

FIG. 7 is a detailed flow chart for obtaining the gas concentration by a Lambert-Beer weighted concentration calculation mode combined with an adaptive piecewise linear temperature compensation mode.

FIG. 8 is a schematic diagram of standard deviations of calibration errors of NDIR gas sensors provided by the present invention.

FIG. 9 is a schematic diagram of fitting curves of NDIR gas sensors provided by the present invention.

### DETAILED DESCRIPTION

The technical solutions in the embodiments of the present invention will be clearly and fully described below in combination with the drawings in the embodiments of the present invention. Apparently, the described embodiments are merely part of the embodiments of the present invention, not all of the embodiments. Based on the embodiments in the present invention, all other embodiments obtained by those ordinary skilled in the art without contributing creative labor will belong to the protection scope of the present invention.

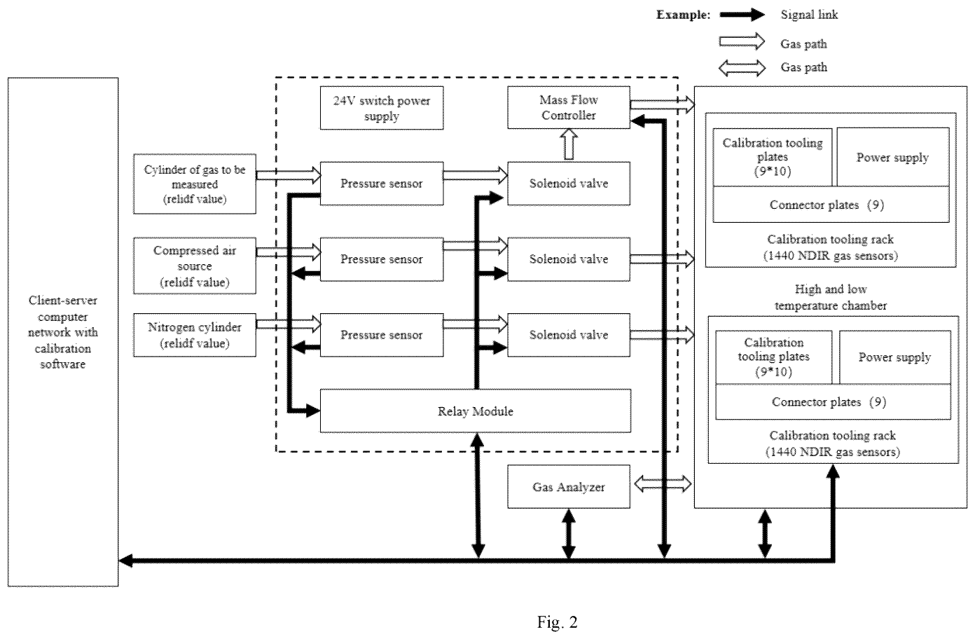

As shown in FIG. 2, an embodiment of the present invention discloses a calibration system of the NDIR gas sensors, which comprises: a client-server computer network with calibration software, a relay module, a gas source group, a mass flow controller, a high and low temperature chamber, a gas analyzer, and a calibration tooling rack group. The client-server computer network is installed with calibration software (developed according to the calibration method, further elaborated upon later) to control and execute broadcasting-style calibration; the calibration tooling rack group is installed in the high and low temperature chamber, and a plurality of calibration tooling plates are installed on the calibration tooling rack group; the NDIR gas sensors to be calibrated are detachably installed on the corresponding calibration tooling plates and connected with the corresponding calibration tooling plates; and a storage medium is arranged inside each NDIR gas sensor and used for storing calibration data.

A calibration tooling rack group having at least two calibration tooling racks is accommodated in the high and low temperature chamber; each of the calibration tooling racks has multiple layers; each layer is provided with at least two connector plates; each connector plate is connected through a golden finger and loaded with a plurality of calibration tooling plates; each calibration tooling plate has a plurality of installation numbers; and each of the NDIR gas sensors is installed on each of the installation numbers one by one.

In the present embodiment, each calibration tooling rack has 9 layers, each layer is provided with 2 connector plates connected in series, and each connector plate is connected by the golden finger and loaded with 5 calibration tooling plates. A data link is established by the calibration tooling plates, the connector plates, and the client-server computer network with calibration software to achieve data communication between the NDIR gas sensors and the client-server computer network with calibration software finally. Each calibration tooling rack is provided with a power supply that powers each NDIR gas sensor through the connector plates.

The client-server computer network with calibration software is connected with the relay module, the mass flow controller, the high and low temperature chamber, the gas analyzer, and each of the calibration tooling plates to realize control or data exchange.

The gas source group comprises a cylinder of the gas to be measured, a compressed air source, and a nitrogen cylinder. Each gas cylinder and each gas source are connected with the high and low temperature chamber through a gas path, respectively, and each gas source outlet pipeline is provided with a pressure sensor and a solenoid valve. The relay module is connected with the pressure sensor and the solenoid valve, respectively. The mass flow controller is equivalent to a switch that can adjust the opening degree and is connected with the solenoid valve through the gas path to adjust the size of the gas flow. The relay module controls the solenoid valve to inject nitrogen or air into the high and low temperature chamber, thereby reducing the gas concentration to be measured in the high and low temperature chamber. At the same time, the relay module reads an input signal of the pressure sensor and feeds the signal back to the client-server computer network with calibration software so as to judge whether the gas source pressure meets the requirements.

The gas analyzer is connected with the high and low temperature chamber for collecting the actual gas concentration to be measured in the high and low temperature chamber.

At the same time, the calibration system of the present invention is further provided with a 24 V switch power supply, and the 24 V switch power supply powers the pressure sensor, the relay module, the solenoid valve, and the mass flow controller.

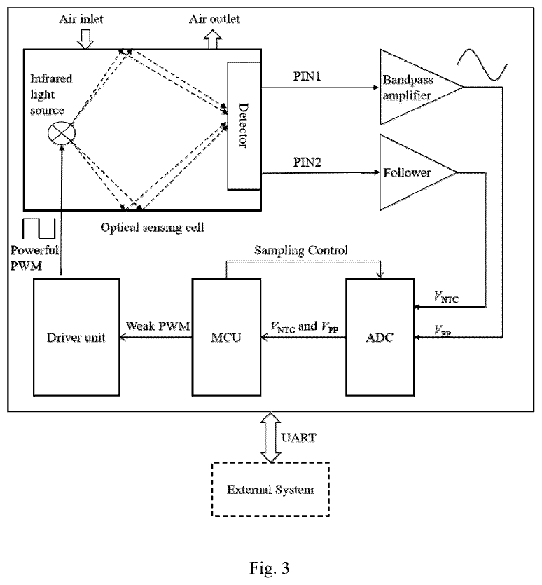

The composition of the NDIR gas sensors is further explained below in combination with FIG. 3.

Each NDIR gas sensor comprises an optical sensing cell, an infrared light source, a detector, a bandpass amplifier, a follower, an ADC module, an MCU, and a driving unit.

A smooth curved mirror is arranged inside the optical sensing cell and used to reflect infrared light and accommodate the gas to be measured. A gas inlet and a gas outlet are arranged on the outer left and right ends of the optical sensing cell. The infrared light source and the detector are oppositely arranged in the optical sensing cell. In the present embodiment, the infrared light source is located on the left side of the optical sensing cell, and the MCU controls the driving unit to output a powerful PWM (Pulse Width Modulation) signal through a weak PWM signal, so that the infrared light source emits infrared light. The infrared light is reflected by the optical sensing cell and transmitted to the detector. The infrared light of a specific wavelength is partially absorbed by the gas to be measured during transmission.

The detector is located on the right side of the optical sensing cell, and an optical filter installed on the surface of the detector allows only the infrared light of the specific wavelength to pass through. A thermopile device and an NTC thermistor are arranged inside the detector, and two pins are led out, i.e., a thermopile pin PIN1 and an NTC thermistor pin PIN2. The thermopile PIN1 is connected with the ADC module through the bandpass amplifier, and the NTC thermistor pin PIN2 is connected with the ADC module through the follower; and the MCU is electrically connected with the ADC module for controlling the sampling of the ADC module.

The detector is sensitive to changes in the infrared light of the specific wavelength, converts the infrared light of the specific wavelength into a voltage signal through the thermopile device, and outputs the voltage signal through the PIN1. Because the voltage signal outputted by the thermopile pin is weak and close to the sawtooth shape and must be amplified and filtered, the bandpass amplifier is needed to amplify the effective signal and filter the noise to output the voltage signal close to a sine wave. Under the control of the MCU, the ADC module collects the maximum value and the minimum value of the voltage signal outputted by the bandpass amplifier to determine a peak-to-peak value. The peak-to-peak value is called the concentration voltage, denoted as V_{PP}, and is directly proportional to the intensity of the infrared light of the specific wavelength incident on the detector. The MCU calculates the gas concentration to be measured through V_{PP} using the specified concentration calculation mode.

The detector, the optical sensing cell, the bandpass amplifier, and other components of the NDIR gas sensor are easily affected by the ambient temperature, resulting in an offset voltage signal. Thus, an NTC thermistor is needed so that the MCU can implement temperature compensation to offset the influence of the ambient temperature. The NTC thermistor inside the detector can convert the ambient temperature into a voltage signal and output the voltage signal through the PIN2. The voltage signal is called the temperature voltage, denoted as V_{NTC}. Under the control of the MCU, the ADC collects temperature voltage and transmits the temperature voltage to the MCU.

The MCU is connected with the calibration tooling plates and the client-server computer network with calibration software successively through a digital communication interface, and receives data capturing instructions, data saving instructions, and the actual gas concentration to be measured in the high and low temperature chamber, broadcasted by the client-server computer network with calibration software.

The storage media are located inside the MCU, and store the concentration voltage Vₚₚ, the temperature voltage V_{NTC}, and the actual gas concentration to be measured under the control of the MCU. When receiving the data capturing instructions, the MCU obtains the current concentration voltage V_{PP}, the temperature voltage V_{NTC}, and the actual gas concentration to be measured; and when receiving the data saving instructions, the concentration voltage Vₚₚ, the temperature voltage V_{NTC}, and the actual gas concentration to be measured are saved to the storage media. In this way, in the actual calibration process, the NDIR gas sensors can directly retrieve the calibration data from the internal storage media, without frequent data interaction with the client-server computer network with calibration software, thereby shortening the calibration time and improving the calibration efficiency.

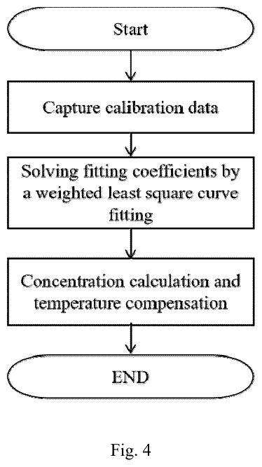

As shown in FIG. 4, the embodiment of the present invention further provides a calibration method for the NDIR gas sensors. The whole process comprises three parts, i.e.:

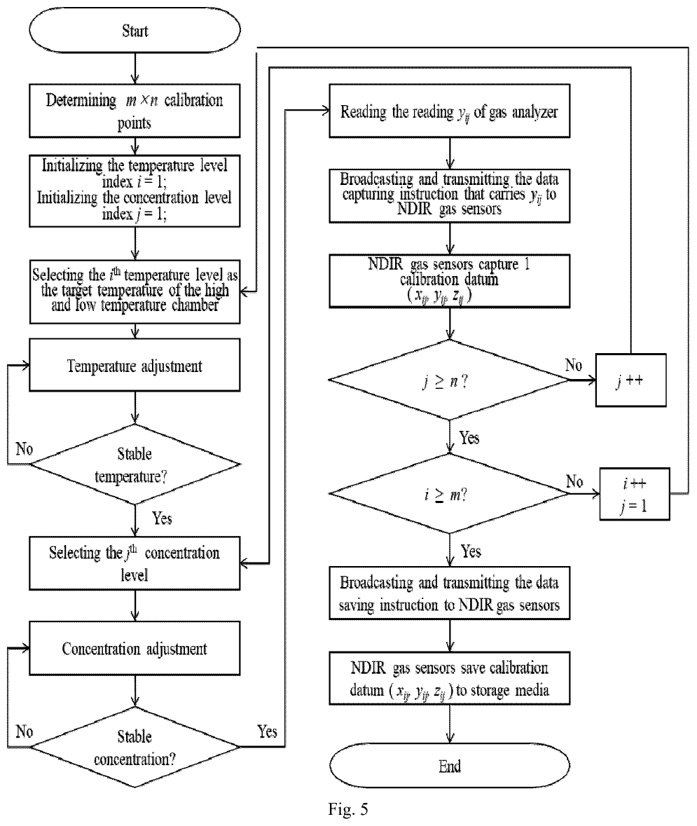

S1. calibration data is captured, as shown in FIG. 5, specifically comprising the following steps:

m calibration temperature levels and n calibration concentration levels are determined, denoted as m×n, according to the working temperature and the range of the NDIR gas sensors, as shown in Table 1. The calibration temperature level is denoted as Tᵢ, and the calibration concentration level is denoted as Cⱼ, wherein i and j are index numbers. i=1, 2, 3, . . . , m corresponds in turn to the calibration temperature levels T₁, T₂, T₃ . . . , Tₘ; j=1, 2, 3, 4, . . . , n corresponds in turn to the calibration concentration levels C₁, C₂, C₃, C₄, . . . , Cₙ, wherein T₁>T₂>T₃>, >Tₘ, and C₁<C₂<C₃<C₄<, . . . , <Cₙ.

TABLE 1
m × n calibration points

| | | j | | | | | |
|---|---|---|---|---|---|---|---|
| | | 1 | 2 | 3 | 4 | . . . | n |
| Index | | Concentration | | | | | |
| i | Temperature | C₁ | C₂ | C₃ | C₄ | . . . | Cₙ |
| 1 | T₁ | (T₁, C₁) | (T₁, C₂) | (T₁, C₃) | (T₁, C₄) | . . . | (T₁, Cₙ) |
| 2 | T₂ | (T₂, C₁) | (T₂, C₂) | (T₂, C₃) | (T₂, C₄) | . . . | (T₂, Cₙ) |
| 3 | T₃ | (T₃, C₁) | (T₃, C₂) | (T₃, C₃) | (T₃, C₄) | . . . | (T₃, Cₙ) |
| . . . | . . . | . . . | . . . | . . . | . . . | . . . | . . . |
| m | Tₘ | (Tₘ, C₁) | (Tₘ, C₂) | (Tₘ, C₃) | (Tₘ, C₄) | . . . | (Tₘ, Cₙ) |

The calibration temperature level T₁ is selected through the client-server computer network with calibration software, and the target temperature level of the high and low temperature chamber is set as T₁ through the instruction. The temperature adjustment function of the high and low temperature chamber is started.

The temperature of the high and low temperature chamber is automatically adjusted and stabilized at T₁.

The client-server computer network with calibration software selects the calibration concentration level C₁.

The client-server computer network with calibration software controls the solenoid valve to close or open the corresponding gas path through the relay module, and controls the gas flow to be measured through the mass flow controller, so as to adjust and stabilize the gas concentration to be measured in the high and low temperature chamber at C₁.

The client-server computer network with calibration software reads the current reading of the gas analyzer, denoted as yᵢⱼ, wherein i=1 and j=1, which can truly reflect the gas concentration to be measured in the high and low temperature chamber.

The client-server computer network with calibration software transmits the data-capturing instruction to the NDIR gas sensors by way of broadcasting, and the instruction contains yᵢⱼ, i=1; and j=1.

After receiving the data capturing instruction, the NDIR gas sensors capture one calibration datum (xᵢⱼ, yᵢⱼ, zᵢⱼ), i=1; j=1; xᵢⱼ is the current Vₚₚ, and zᵢⱼ is the current V_{NTC}.

The temperature of the high and low temperature chamber remains unchanged. The same process is repeated. The NDIR gas sensors successively capture the calibration data (xᵢⱼ, yᵢⱼ, zᵢⱼ) when the gas concentration to be measured in the high and low temperature chamber is stabilized at C₂, C₃, C₄, . . . , Cₙ, wherein i=1; and j=2, 3, 4, . . . , n. Then, the calibration data (xᵢⱼ, yᵢⱼ, zᵢⱼ) when the calibration temperature level is T₁ has been successfully captured, wherein i=1; and j=12, 3, 4, . . . , n.

The same process is repeated. The NDIR gas sensors successively capture the calibration data (xᵢⱼ, yᵢⱼ, zᵢⱼ) when the temperature of the high and low temperature chamber is stabilized at T₂, T₃, . . . , Tₘ, wherein i=2, 3, . . . , m; and j=1, 2, 3, 4, . . . , n.

Then, the calibration data (xᵢⱼ, yᵢⱼ, zᵢⱼ) corresponding to all the calibration points has been successfully captured, wherein i=1 2, 3, . . . , m; and j=1, 2, 3, 4, . . . , n.

The client-server computer network with calibration software broadcasts and transmits the data-saving instructions to the NDIR gas sensors.

After receiving the data saving instruction, the NDIR gas sensors save the calibration data (xᵢⱼ, yᵢⱼ, zᵢⱼ) to the internal storage medium, i=1 2, 3, . . . , m; and j=1, 2, 3, 4, . . . , n. Then, the process of capturing the calibration data is complete.

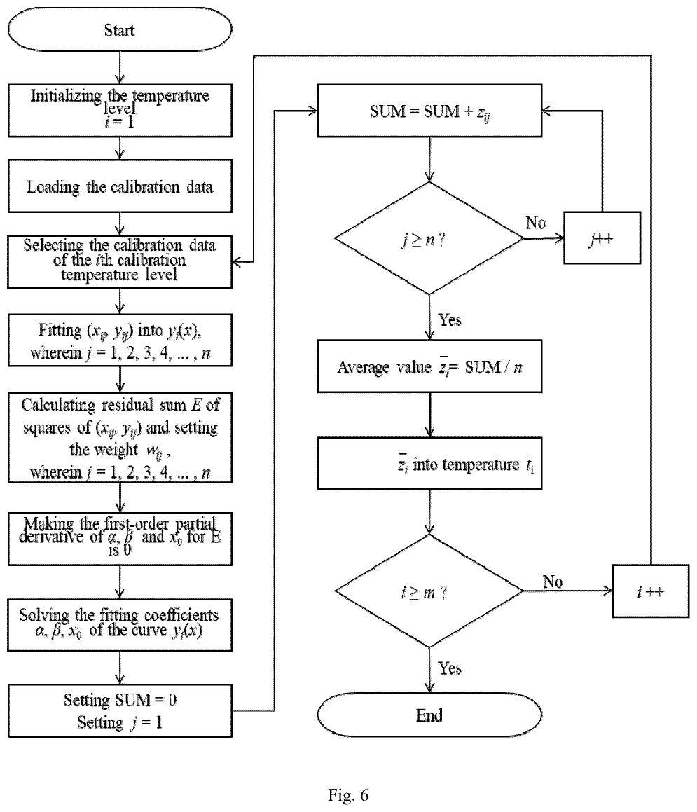

S2. fitting curves are solved by weighted least squire curve fitting, as shown in FIG. 6:

The formula of the Lambert-Beer fitting curve is shown in formula (1):

$$I = {I}_{0} \cdot {e}^{- α {C}^{β}} \qquad ( 1 )$$

In formula (1), I represents the transmitted light intensity (in W/m²) of the light after passing through the gas to be measured; I₀ represents the incident light intensity (that is, the initial signal intensity when the gas concentration to be measured is 0, in W/m²); C is the gas concentration to be measured; a represents the comprehensive absorption coefficient of the gas to be measured in a specific band; and β is a correction factor that depends on the optical structure and the characteristics of the optical filter.

Formula (1) is derived to obtain the expression of the gas concentration to be measured C, as shown in formula (2).

$$C = {[ - \frac{ln ( I / {I}_{0} )}{α} ]}^{\frac{1}{β}} \qquad ( 2 )$$

I and I₀ are replaced with x and x₀, respectively, as shown in formula (3).

$$\frac{I}{{I}_{0}} = \frac{x}{{x}_{0}} \qquad ( 3 )$$

In formula (3), x is V_{PP} corresponding to the emergent light intensity I, and x₀ is V_{PP} corresponding to the incident light intensity I₀.

By combining formula (1), formula (2), and formula (3), the gas concentration to be measured C is expressed by y(x), and the expression after Lambert-Beer transformation is obtained, as shown in formula (4).

$$y ( x ) = {[ - \frac{ln ( x / {x}_{0} )}{α} ]}^{\frac{1}{β}} \qquad ( 4 )$$

Next, the process of solving m-fitting curves of formula (4) by the weighted least square curve fitting is introduced.

The NDIR gas sensors load the calibration data (xᵢⱼ, yᵢⱼ, zᵢⱼ) from the internal storage media, wherein i=1 2, 3, . . . , m; and j=1, 2, 3, 4, . . . , n.

The NDIR gas sensors extract (xᵢⱼ, yᵢⱼ) from the calibration data at the calibration temperature level of T₁, wherein i=1; and j=1, 2, 3, 4, . . . , n.

The NDIR gas sensors fit (xᵢⱼ, yᵢⱼ) as a curve of formula (4), denoted as y₁(x), wherein i=1; and j=1, 2, 3, 4, . . . , n.

The NDIR gas sensors calculate the residual sum E of squares of (xᵢⱼ, yᵢⱼ) according to the principle of a weighted least square curve fitting and set a weight wᵢⱼ, wherein i=1; and j=1, 2, 3, 4, . . . , n. The weight wᵢⱼ is set according to the gas concentration to be measured, that is, wᵢⱼ=1/yᵢⱼ². When wᵢⱼ=1, no weight is set. When wᵢⱼ=1/yᵢⱼ², the weight is related to the gas concentration to be measured. The lower the gas concentration to be measured, the greater the weight.

According to an extremum principle, the first-order partial derivative of α, α, and x₀ in the above formula is 0, so that the residual sum E of squares is minimum.

The NDIR gas sensors solve the fitting coefficients α, β, and x₀ corresponding to the fitting curve y₁(x).

The NDIR gas sensors calculate the average value of zᵢⱼ, denoted as z̅₁, wherein i=1; and j=1, 2, 3, 4, . . . n.

The NDIR gas sensors convert z̅₁ to temperature, denoted as t₁, in ° C.

According to the same process, the NDIR gas sensors solve and calculate the fitting curves y₂(x), y₃(x), . . . , yₘ(x) and the corresponding temperatures t₂, t₃, . . . , tₘ successively when the calibration temperature levels are T₂, T₃, . . . , Tₘ.

Then, m fitting curves y₁(x), y₂(x), y₃(x), . . . , yₘ(x) and the corresponding temperatures t₁, t₂, t₃, . . . , tₘ have been successfully solved, wherein t₁>t₂>t₃>, . . . , >tₘ. They will be used for implementing the concentration calculation mode and the temperature compensation mode in the next process

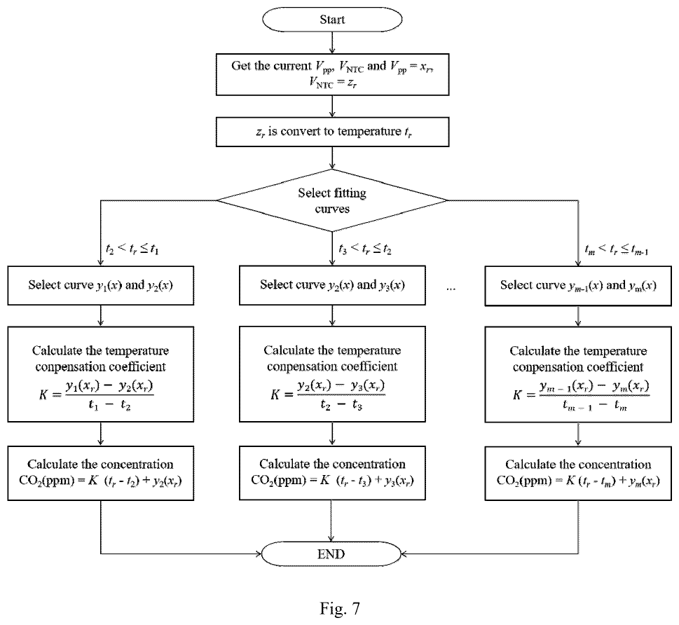

S3. The Lambert-Beer weighted concentration calculation mode combined with the adaptive piecewise linear temperature compensation mode, as shown in FIG. 7:

t₁, t₂, t₃, . . . , tₘ are divided into m−1 temperature intervals, which are: [tₘ, tₘ₋₁], . . . , [t₃, t₂], [t₂, t₁].

To calculate the gas concentration to be measured (denoted as yᵣ), the NDIR gas sensors obtain the current data V_{PP}=xᵣ and V_{NTC}=zᵣ.

zᵣ is converted to temperature, denoted as tᵣ, in ° C., and tᵣ∈[tₘ, t₁].

The temperature interval where tᵣ is located is confirmed, and adjacent fitting curves are selected to calculate the gas concentration to be measured as follows:

When tᵣ∈[tₘ, tₘ₋₁], the fitting curves yₘ₋₁(x) and yₘ(x) are selected, xᵣ is substituted into the curves to calculate yₘ₋₁(xᵣ) and yₘ(xᵣ), respectively, and the temperature compensation coefficient K and the concentration Yᵣ are calculated by formula (5) and formula (6):

$$K = \frac{[ {y}_{m - 1} ( {x}_{r} ) - {y}_{m} ( {x}_{r} ) ]}{{t}_{m - 1} - {t}_{m}} \qquad ( 5 )$$

$${y}_{r} = K ( {t}_{r} - {t}_{m} ) + {y}_{m} ( {x}_{r} ) \qquad ( 6 )$$

When tᵣ∈[t₃, t₂], the fitting curves y₂(x) and y₃(x) are selected, and K and yᵣ are calculated by formula (7) and formula (8):

$$K = \frac{[ {y}_{2} ( {x}_{r} ) - {y}_{3} ( {x}_{r} ) ]}{{t}_{2} - {t}_{3}} \qquad ( 7 )$$

$${y}_{r} = K ( {t}_{r} - {t}_{3} ) + {y}_{3} ( {x}_{r} ) \qquad ( 8 )$$

When tᵣ∈[t₂, t₁], the fitting curves y₁(x) and y₂(x) are selected, and K and yᵣ are calculated by formula (9) and formula (10):

$$K = \frac{[ {y}_{2} ( {x}_{r} ) - {y}_{3} ( {x}_{r} ) ]}{{t}_{1} - {t}_{2}} \qquad ( 9 )$$

$${y}_{r} = K ( {t}_{r} - {t}_{2} ) + {y}_{2} ( {x}_{r} ) \qquad ( 10 )$$

Then, the process of concentration calculation and temperature compensation is complete.

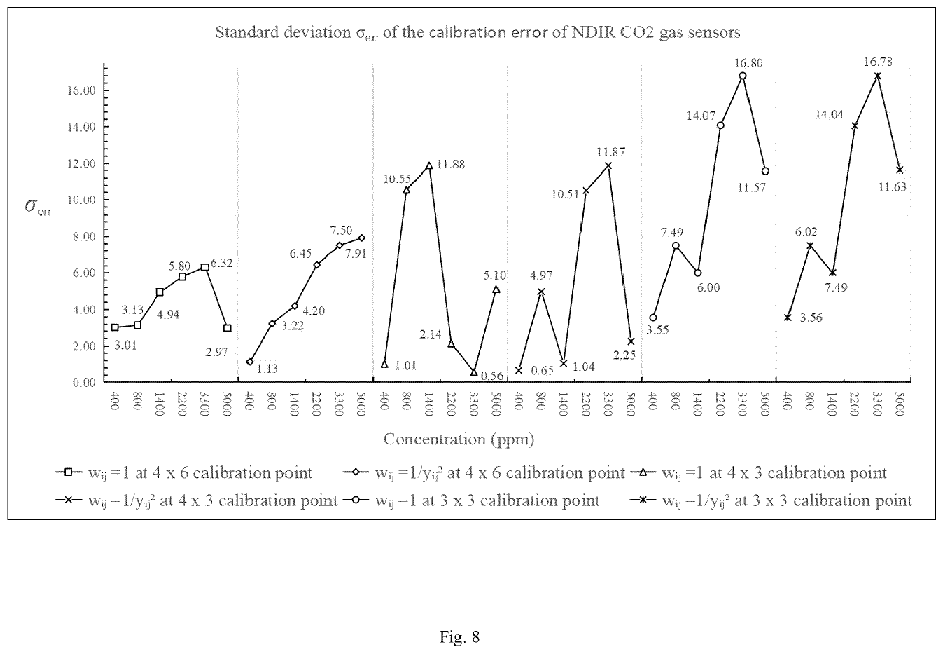

As shown in FIG. 8, in order to further illustrate the accuracy of the calibration method of the present invention, when the calibration points are reduced appropriately, the calibration data (xᵢⱼ, yᵢⱼ, zᵢⱼ) are obtained from the production data of 1,000 NDIR CO₂ (carbon dioxide) gas sensors. Then, the measurement results are calculated in combination with the calibration method of the present invention. Finally, taking the calibration concentration level Cⱼ as a reference, the standard deviation (denoted as σₑᵣᵣ) of the calibration error of the measurement results is calculated to represent the accuracy of the NDIR CO₂ gas sensors. When there are 4 calibration temperature levels and 6 calibration concentration levels (denoted as 4×6, the same as below), it takes 16 hours to calibrate a batch of products. Then, the calibration error of the measurement results conforms to the specification. Accordingly, when the calibration points are reduced to 4×3 and 3×3, the calibration error of the measurement results still conforms to the specification: 3σₑᵣᵣ≤(Cⱼ×5%+50) ppm. After the calibration points are reduced to 4×3 and 3×3, it takes only 14 hours and 10.5 hours to calibrate a batch of products, which means that the calibration efficiency is significantly improved.

In addition, when 4×6 calibration points are used, σₑᵣᵣ is 3.01 and 1.13 at wᵢⱼ=1 and wᵢⱼ=1/yᵢⱼ²; and when 4×3 calibration points are used, σₑᵣᵣ is 1.01 and 0.65 at wᵢⱼ=1 and wᵢⱼ=1/yᵢⱼ², thereby indicating that a higher weight within the low concentration range effectively improves the accuracy of the NDIR CO₂ gas sensors within the low concentration range. It can be seen that compared with the conventional Lambert-Beer fitting, the calibration method of the present invention further improves the accuracy of the NDIR gas sensors within the low concentration range.

Because the calibration method of the NDIR gas sensors (such as NDIR CO gas sensors, NDIR CH₄ gas sensors, and NDIR CO₂ gas sensors) are the same, the present invention takes the NDIR CO₂ gas sensors and the 4×6 calibration points as an example to explain the above calibration method in detail.
S101. according to the operating temperature and the range, 4×6 calibration points are determined, and the corresponding index numbers i,j are set, as shown in Table 2.
S102. the client-server computer network with calibration software selects the calibration temperature level of 50° C.
S103. the client-server computer network with calibration software sets the target temperature level of the high and low temperature chamber to 50° C. and starts the temperature adjustment function of the high and low temperature chamber through instructions.
S104. the temperature of the high and low temperature chamber is automatically adjusted and stabilized at 50° C.
S105. the client-server computer network with calibration software selects the calibration concentration level of 400 ppm.

TABLE 2
4 × 6 calibration points

| | | j | | | | | |
|---|---|---|---|---|---|---|---|
| | | 1 | 2 | 3 | 4 | 5 | 6 |
| Index | Temperature | Concentration (ppm) | | | | | |
| i | (° C.) | 400 | 800 | 1400 | 2200 | 3300 | 5000 |
| 1 | 50 | (50, 400) | (50, 800) | (50, 1400) | (50, 2200) | (50, 3300) | (50, 5000) |
| 2 | 25 | (25, 400) | (25, 800) | (25, 1400) | (25, 2200) | (25, 3300) | (25, 5000) |
| 3 | 5 | (5, 400) | (5, 800) | (5, 1400) | (5, 2200) | (5, 3300) | (5, 5000) |
| 4 | −10 | (−10, 400) | (−10, 800) | (−10, 1400) | (−10, 2200) | (−10, 3300) | (−10, 5000) |

S106. the client-server computer network with calibration software controls the solenoid valve to close or open the corresponding gas path through the relay module, and controls the flow of the CO₂ gas through the mass flow controller, so as to adjust and stabilize the CO₂ gas concentration in the high and low temperature chamber at 400 ppm.
S107. the client-server computer network with calibration software reads the current reading of the gas analyzer, denoted as yᵢⱼ, wherein i=1 and j=1, which can truly reflect the CO₂ gas concentration in the high and low temperature chamber.
S108. the client-server computer network with calibration software transmits the data-capturing instruction to the NDIR CO₂ gas sensors by way of broadcasting, and the instruction contains yᵢⱼ=1; and j=1.
S109. after receiving the data capturing instruction, the NDIR CO₂ gas sensors capture one calibration datum (xᵢⱼ, yᵢⱼ, zᵢⱼ), i=1; j=1; x₁₁ is the current V_{PP}, and zᵢⱼ is the current V_{NTC}.

S110. the temperature of the high and low temperature chamber remains unchanged. The same process as S105 to S109 is repeated. The NDIR CO₂ gas sensors successively capture the calibration data (xᵢⱼ, yᵢⱼ, zᵢⱼ) when the CO₂ gas concentration in the high and low temperature chamber is stabilized at 800 ppm, 1400 ppm, 2200 ppm, 3300 ppm, and 5000 ppm, wherein i=1; and j=2, 3, 4, 5, 6.

S111. then, the calibration data (xᵢⱼ, yᵢⱼ, zᵢⱼ) when the calibration temperature level is 50° C. has been successfully captured, wherein i=1; and j=1 2, 3, 4, 5, 6.

S112. the same process as S102 to S110 is repeated; and the NDIR CO₂ gas sensors successively capture the calibration data (xᵢⱼ, yᵢⱼ, zᵢⱼ) when the temperature of the high and low temperature chamber is stabilized at 25° C., 5° C., and −10° C., wherein i=2, 3, 4; and j=1 2, 3, 4, 5, 6.

Then, the calibration data (xᵢⱼ, yᵢⱼ, zᵢⱼ) corresponding to all the calibration points has been successfully captured, wherein i=1 2, 3, 4; and j=1, 2, 3, 4, 5, 6.
S113. the client-server computer network with calibration software broadcasts and transmits the data-saving instructions to the NDIR CO₂ gas sensors.
S114. after receiving the data saving instruction, the NDIR CO₂ gas sensors save the calibration data (xᵢⱼ, yᵢⱼ, zᵢⱼ) to the internal storage medium, i=1, 2, 3, 4; and j=1, 2, 3, 4, 5, 6.
S115. The NDIR CO₂ gas sensors load the calibration data (xᵢⱼ, yᵢⱼ, zᵢⱼ) from the internal storage media, wherein i=1, 2, 3, 4; and j=1 2, 3, 4, 5, 6.
S201. the NDIR CO₂ gas sensors extract (xᵢⱼ, yᵢⱼ) from the calibration data at the calibration temperature level of 50° C., wherein i=1; and j=1, 2, 3, 4, 5, 6.
S202. the NDIR CO₂ gas sensors fit (xᵢⱼ, yᵢⱼ) as a curve of formula (4), denoted as yᵢⱼ(x), wherein i=1; and j=1, 2, 3, 4, 5, 6.
S203. the NDIR CO₂ gas sensors calculate the residual sum E of squares of (xᵢⱼ, yᵢⱼ) according to the principle of a weighted least square curve fitting and set a weight wᵢⱼ, wherein i=1; and j=1, 2, 3, 4, 5, 6, as shown in formula (11).

$$E = {\sum }_{j = 1}^{6} {{w}_{ij} [ y ( {x}_{ij} ) - {y}_{ij} ]}^{2} = {\sum }_{j = 1}^{6} {{w}_{ij} [ {( \frac{- ln ( {x}_{ij} / {x}_{0} )}{α} )}^{\frac{1}{β}} - {y}_{ij} ]}^{2} \qquad ( 11 )$$

S204. according to an extremum principle, the first-order partial derivative of α, β, and x₀ in the formula (11) shall be 0 in order to make the residual sum E of squares minimum, wherein i=1; and j=1, 2, 3, 4, 5, 6, as shown in formula (12), formula (13), and formula (14).

$$\frac{\partial E}{\partial a} = - \frac{2}{β} {\sum }_{j = 1}^{6} {{w}_{ij} [ \frac{{[ ln ( {x}_{ij} / {x}_{0} ) ]}^{2}}{{α}^{2 + β}} ]}^{\frac{1}{β}} + \frac{2}{β} {\sum }_{j = 1}^{6} {w}_{ij} {{y}_{ij} [ - \frac{ln ( {x}_{ij} / {x}_{0} )}{{α}^{1 + β}} ]}^{\frac{1}{β}} = 0 \qquad ( 12 )$$

$$\frac{\partial E}{\partial β} = - \frac{2}{{β}^{2}} {\sum }_{j = 1}^{6} {{w}_{ij} [ - \frac{ln ( {x}_{ij} / {x}_{0} )}{α} ]}^{\frac{2}{β}} * ln [ - \frac{ln ( {x}_{ij} / {x}_{0} )}{α} ] + \frac{2}{{β}^{2}} {\sum }_{j = 1}^{6} {w}_{ij} {{y}_{ij} [ - \frac{ln ( {x}_{ij} / {x}_{0} )}{α} ]}^{\frac{1}{β}} * ln [ - \frac{ln ( {x}_{ij} / {x}_{0} )}{α} ] = 0 \qquad ( 13 )$$

$$\frac{\partial E}{\partial {x}_{0}} = - \frac{2}{αβ {x}_{0}} {\sum }_{j = 1}^{6} {{w}_{ij} [ - \frac{ln ( {x}_{ij} / {x}_{0} )}{α} ]}^{\frac{2 - β}{β}} + \frac{2}{αβ {x}_{0}} {\sum }_{j = 1}^{6} {w}_{ij} {{y}_{ij} [ - \frac{ln ( {x}_{ij} / {x}_{0} )}{α} ]}^{\frac{1 - β}{β}} = 0 \qquad ( 14 )$$

S205. formula (12), formula (13), and formula (14) are simplified to obtain formula (15), formula (16), and formula (17), wherein i=1; and j=1, 2, 3, 4, 5, 6.

$${\sum }_{j = 1}^{6} {{w}_{ij} [ - ln ( {x}_{ij} / {x}_{0} ) ]}^{\frac{2}{β}} = {α}^{\frac{1}{β}} {\sum }_{j = 1}^{6} {w}_{ij} {{y}_{ij} [ - ln ( {x}_{ij} / {x}_{0} ) ]}^{\frac{1}{β}} \qquad ( 15 )$$

$$\begin{matrix}{\sum }_{j = 1}^{6} {{w}_{ij} [ - \frac{ln ( {x}_{ij} / {x}_{0} )}{α} ]}^{\frac{2}{β}} * ln [ - \frac{ln ( {x}_{ij} / {x}_{0} )}{α} ] \\ = {\sum }_{j = 1}^{6} {w}_{ij} {{y}_{ij} [ - \frac{ln ( {x}_{ij} / {x}_{0} )}{α} ]}^{\frac{1}{β}} * ln [ - \frac{ln ( {x}_{ij} / {x}_{0} )}{α} ]\end{matrix} \qquad ( 16 )$$

$${\sum }_{j = 1}^{6} {{w}_{ij} [ - ln ( {x}_{ij} / {x}_{0} ) ]}^{\frac{2 - β}{β}} = {α}^{\frac{1}{β}} {\sum }_{j = 1}^{6} {w}_{ij} {{y}_{ij} [ - ln ( {x}_{ij} / {x}_{0} ) ]}^{\frac{1 - β}{β}} \qquad ( 17 )$$

S206. the NDIR CO₂ gas sensors solve the fitting coefficients α, β, and x₀ of the fitting curve y₁(x) by formula (15), formula (16), and formula (17).
S207. the NDIR CO₂ gas sensors calculate the average value of zᵢⱼ, denoted as z̅₁, wherein i=1; and j=1, 2, 3, 4, 5, 6, as shown in formula (18).

$$\overline{{z}_{1}} = \frac{1}{6} {\sum }_{j = 1}^{6} {z}_{ij} \qquad ( 18 )$$

S208. the NDIR CO₂ gas sensors convert zi to temperature, denoted as t₁, in ° C.
S209. the same process as S201 to S208 is repeated, and the NDIR CO₂ gas sensors solve and calculate the fitting curves y₂(x), y₃(x), and y₄(x) and the corresponding temperatures t₂, t₃, and t₄ successively when the calibration temperature levels are 25° C., 5° C., and −10° C. The relationship among t₁, t₂, t₃, and t₄ is: t₁>t₂>₃>t₄.

Then, four fitting curves y₁(x), y₂(x), y₃(x), and y₄(x) and the corresponding temperatures t₁, t₂, t₃, and t₄ have been successfully solved, wherein t₁>t₂>t₃>t₄.

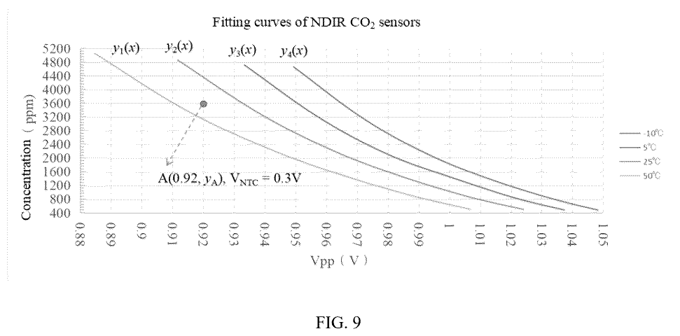

As shown in FIG. 9, with Vₚₚ as the x-axis and concentration as the y-axis, the four fitted curves y₁(x), y₂(x), y₃(x), and y₄(x) correspond to 50° C., 25° C., 5° C., and −10° C., respectively. Note: This step begins to illustrate how to obtain the value of the CO₂ gas concentration to be measured by this algorithm according to the measured V_{PP} and V_{NTC}.
S301. 50° C., 25° C., 5° C., and −10° C. are divided into three temperature intervals [−10, 5], [5, 25], and [25, 50].
S302. the NDIR CO₂ gas sensors obtain real-time data V_{PP}=0.92 V and V_{NTC}=0.3 V.
S303. the NDIR gas sensors convert V_{NTC}=0.3 V into temperature to obtain 42.9° C., which falls in the temperature interval [25° C., 50° C.] and is represented by point A in FIG. 9. Then, the CO₂ gas concentration to be measured y_{A} is calculated by formula (19):

$${y}_{A} = \frac{[ {y}_{1} ( 0.92 ) - {y}_{2} ( 0.92 ) ] ( 42.9 - 25 )}{50 - 25} + {y}_{2} ( 0.92 ) \qquad ( 19 )$$

Each embodiment in the description is described in a progressive way. The difference between each embodiment and the others is the focus of the explanation. The same and similar parts among all the embodiments can be referred to each other. For a device disclosed by the embodiments, because the device corresponds to a method disclosed by the embodiments, the device is simply described. Refer to the description of the method part for the related part.

The above description of the disclosed embodiments enables those skilled in the art to realize or use the present invention. Many modifications to these embodiments will be apparent to those skilled in the art. The general principle defined herein can be realized in other embodiments without departing from the spirit or scope of the present invention. Therefore, the present invention will not be limited to these embodiments shown herein, but will conform to the widest scope consistent with the principle and novel features disclosed herein.

What is claimed is:

1. A calibration system for NDIR gas sensors, comprising: a client-server computer network with calibration software, a relay module, a gas source group, a mass flow controller, a high and low temperature chamber, a gas analyzer, and a calibration tooling rack group, wherein the client-server computer network is installed with calibration software (developed according to the calibration method) to control and execute broadcasting-style calibration; the calibration tooling rack group is installed in the high and low temperature chamber, and a plurality of calibration tooling plates are installed on the calibration tooling rack group; the NDIR gas sensors to be calibrated are detachably installed on the corresponding calibration tooling plates and connected with the corresponding calibration tooling plates; and a storage medium is arranged inside each NDIR gas sensor and used for storing calibration data;
each gas source in the gas source group is connected with the high and low temperature chamber, respectively, through a gas path, and each gas path is provided with a pressure sensor and a solenoid valve; the relay module is connected with the pressure sensor and the solenoid valve respectively; and the mass flow controller is connected with the solenoid valve through the gas path for adjusting the size of the gas flow;
the gas analyzer is connected with the high and low temperature chamber for collecting the actual gas concentration to be measured in the high and low temperature chamber;
the client-server computer network with calibration software is connected with the relay module, the mass flow controller, the high and low temperature chamber, the gas analyzer, and each of the calibration tooling plates to realize control or data exchange.

2. The calibration system for NDIR gas sensors according to claim 1, wherein a calibration tooling rack group having at least two calibration tooling racks is accommodated in the high and low temperature chamber; each of the calibration tooling racks has multiple layers; each layer is provided with at least two connector plates; each connector plate is connected through a golden finger and loaded with a plurality of calibration tooling plates; each calibration tooling plate has a plurality of installation numbers; and each of the NDIR gas sensors is installed on each of the installation numbers one by one.

3. The calibration system for NDIR gas sensors according to claim 2, wherein a plurality of tooling power supplies are also installed on the calibration tooling racks, and each of the tooling power supplies powers the plurality of NDIR gas sensors.

4. The calibration system for NDIR gas sensors according to claim 1, wherein the calibration system further comprises a 24 V switch power supply, and the 24 V switch power supply powers the pressure sensor, the solenoid valve, the relay module, and the mass flow controller.

5. The calibration system for NDIR gas sensors according to claim 1, wherein the gas source group comprises a cylinder of the gas to be measured, a compressed air source, and a nitrogen cylinder.

6. The calibration system for NDIR gas sensors according to claim 2, wherein each NDIR gas sensor comprises an optical sensing cell, an infrared light source, a detector, a bandpass amplifier, a follower, an ADC module, an MCU, and a driving unit;
the optical sensing cell is provided with an air inlet and an air outlet; the infrared light source and the detector are relatively arranged in the optical sensing cell; the driving unit drives the infrared light source to emit light under the control of the MCU; and the optical sensing cell reflects the infrared light emitted by the infrared light source for entering the detector;
a thermopile device and an NTC thermistor are arranged inside the detector, and two pins are led out, i.e., a thermopile pin PIN1 and an NTC thermistor pin PIN2; the thermopile PIN1 is connected with the ADC module through the bandpass amplifier, and the NTC thermistor pin PIN2 is connected with the ADC module through the follower; the MCU is connected with the ADC module for controlling the sampling of the ADC module; and the ADC module measures the peak-to-peak value of a voltage signal outputted by the bandpass amplifier as a concentration voltage V_{PP}, and captures a voltage signal outputted by the follower as a temperature voltage V_{NTC};
the MCU is connected with the calibration tooling plates and the client-server computer network with calibration software successively through a digital communication interface, and receives data capturing instructions, data saving instructions, and the actual gas concentration to be measured in the high and low temperature chamber, broadcasted by the client-server computer network with calibration software;
the storage media are located inside the MCU, and store the concentration voltage V_{PP}, the temperature voltage V_{NTC}, and the actual gas concentration to be measured under the control of the MCU.

7. A calibration method for NDIR gas sensors, which is applicable to the calibration system for the NDIR gas sensors of claim 1, comprising the following steps:
determining m calibration temperature levels and n calibration concentration levels according to the working temperature and the range of the NDIR gas sensors;
capturing, by the NDIR gas sensors, calibration data (xᵢⱼ, yᵢⱼ, zᵢⱼ) at each calibration concentration level at any calibration temperature level, wherein i=1, 2, 3, . . . , m; j=1, 2, 3, 4, . . . , n; xᵢⱼ is the concentration voltage V_{PP} currently outputted by the NDIR gas sensors; zᵢⱼ is the temperature voltage V_{NTC} currently outputted by the NDIR gas sensors; and yᵢⱼ is the actual gas concentration to be measured in the high and low temperature chamber collected by the gas analyzer;
broadcasting and transmitting, by the client-server computer network with calibration software, the data saving instructions to each NDIR gas sensor, and saving, by the NDIR gas sensors, the calibration data to the internal storage media;
extracting, by the NDIR gas sensors, (xᵢⱼ, yᵢⱼ) from the calibration data with the calibration temperature level of T₁, wherein i=1; and j=1, 2, 3, 4, . . . , n;
fitting (xᵢⱼ, yᵢⱼ) as a curve, denoted as yᵢⱼ(x), calculating the residual sum E of squares of (xᵢⱼ, yᵢⱼ) according to the principle of a weighted least square curve fitting and setting a weight wᵢⱼ, wherein i=1; and j=1, 2, 3, 4, . . . , n;
solving fitting coefficients α, β, and x₀ corresponding to the fitting curve yᵢⱼ(x) and corresponding temperature t₁;
solving, by the NDIR gas sensors, fitting curves y₂(x), y₃(x), . . . , yₘ(x) and corresponding temperatures t₂, t₃, . . . , tₘ successively at the calibration temperature levels of T₂, T₃, . . . , Tₘ according to the same process;
dividing the temperatures t₁, t₂, t₃, . . . , tₘ into m−1 temperature intervals, which are: [tₘ, tₘ₋₁], . . . [t₃, t₂], [t₂, t₁];
acquiring current data Vₚₚ=xᵣ and V_{NTC}=zᵣ by the NDIR gas sensors, and converting zᵣ into temperature, denoted as tᵣ, tᵣ∈[tₘ, t₁];
confirming the temperature interval where tᵣ is located, and selecting adjacent fitting curves to calculate the gas concentration to be measured yᵣ and the temperature compensation coefficient K.

8. The calibration method for NDIR gas sensors according to claim 7, wherein the solving process of the fitting coefficients α, β, and x₀ corresponding to the fitting curve y₁(x) is as follows:
calculating the residual sum E of squares of (xᵢⱼ, yᵢⱼ) according to the following formula:
$$E = {\sum }_{j = 1}^{n} {{w}_{ij} [ y ( {x}_{ij} ) - {y}_{ij} ]}^{2} = {\sum }_{j = 1}^{n} {{w}_{ij} [ {( \frac{- ln ( {x}_{ij} / {x}_{0} )}{α} )}^{\frac{1}{β}} - {y}_{ij} ]}^{2}$$
according to an extremum principle, the first-order partial derivative of α, β, and x₀ in the above formula is 0, so that the residual sum E of squares is minimum, wherein i=1; and j=1, 2, 3, 4, . . . , n;
solving the fitting coefficients α, β, and x₀ of the fitting curve y₁(x) for implementing the Lambert-Beer weighted concentration calculation mode.

9. The calibration method of the NDIR gas sensors according to claim 7, wherein the calculation of temperature t₁ is as follows:
at the calibration temperature level T₁, calculating the average value of the temperature voltage zᵢⱼ outputted by the NDIR gas sensors at each calibration concentration level, denoted as z̅₁,
$$\overline{{z}_{1}} = \frac{1}{n} {\sum }_{j = 1}^{n} {z}_{ij} ,$$
wherein i=1; and j=1, 2, 3, 4, . . . , n;
converting z̅₁ into temperature, denoted as t₁.

10. The calibration method for the NDIR gas sensors according to claim 7, wherein the Lambert-Beer weighted concentration calculation mode combined with the adaptive piecewise linear temperature compensation mode is implemented as follows:
when the temperature tᵣ∈[tₘ, tₘ₋₁], the fitting curves yₘ₋₁(x) and yₘ(x) are selected, xᵣ is substituted into the curves to calculate yₘ₋₁(xᵣ) and yₘ(xᵣ), respectively, and the temperature compensation coefficient K and the gas concentration to be measured yᵣ are calculated according to the following formulas:
$$\begin{matrix}K = \frac{[ {y}_{m - 1} ( {x}_{r} ) - {y}_{m} ( {x}_{r} ) ]}{{t}_{m - 1} - {t}_{m}} \\ {y}_{r} = K ( {t}_{r} - {t}_{m} ) + {y}_{m} ( {x}_{r} ) .\end{matrix}$$
